(19) United States
(12) Patent Application Publication
(10) Pub. No.: US 2024/0024486 A1
(43) Pub. Date: Jan. 25, 2024

(54) PHARMACEUTICAL COMPOSITION FOR TOPICAL USE COMPRISING A POLYMER IN THE FORM OF A PULVERULENT SOLID AS THICKENING AGENT

(71) Applicant: SOCIETE D'EXPLOITATION DE PRODUITS POUR LES INDUSTRIES CHIMIQUES SEPPIC, PARIS CEDEX 07 (FR)

(72) Inventors: Miruna BODOC, CASTRES (FR); Stéphane MONTEILLET, CASTRES (FR)

(21) Appl. No.: 18/256,869

(22) PCT Filed: Nov. 30, 2021
(86) PCT No.: PCT/EP2021/083622

§ 371 Date: Jun. 9, 2023

(30) Foreign Application Priority Data
Dec. 14, 2020 (FR) 2013175

(51) Int. Cl. A61K 47/34; A61K 45/06

(52) U.S. Cl. CPC A61K 47/34; A61K 45/06

(57) ABSTRACT

Disclosed is a pharmaceutical composition (F) for topical use, comprising at least one active pharmaceutical ingredient and, as thickening agent, a polymer (P) in the form of a pulverulent solid consisting of monomeric units derived from partially or totally salified glutamic acid (GA) and monomer units of at least one crosslinking agent (AR) having at least two glycidyl functions.

The present invention relates to a pharmaceutical composition (F) for topical use comprising, as thickening agent, a polymer (P) in the form of a pulverulent solid, and to the process for preparing said pharmaceutical composition (F).

Polymers are widely used today in pharmaceutical compositions for topical use and represent the second most widely used family of products in compositions of this type. Pharmaceutical compositions for topical use contain polar phases, for instance phases consisting of water, and in most cases require the use of rheology modifiers, for instance polymers, to increase the viscosity of these polar phases, and also to impart well-defined rheological behavior. Among the polymers which modify the rheology of polar phases, mention may be made of natural polymers, for instance polysaccharides based on monosaccharides or polysaccharides based on monosaccharide derivatives or else synthetic polymers, of linear or branched, crosslinked or noncrosslinked, anionic or cationic, or amphiphilic, polyelectrolyte type. Predominantly present on the market, synthetic polymers have the property of being deployed, in the polar phase, under the effect of electrostatic repulsions due to the presence of charges (negative and/or positive) on the linear or branched, crosslinked or noncrosslinked polymer backbone. These rheology modifiers bring both an increase in the viscosity of the polar phase, and also a certain consistency and/or a stabilizing effect imparted upon the pharmaceutical composition for topical use.

In order to meet the needs of formulators and to improve performance, various recent scientific studies have reported the development of new, innovative and varied polymeric systems. Thus, the polymers used in the detergent industries can play a functional role as film-forming agents, rheology modifiers, enabling stabilization of the fatty phases in emulsions of the water-in-oil type and of the oil-in-water type, stabilization of solid particles (pigments and fillers) or as agents having an impact on the appearance of the formula (transparency, translucency, opacity).

The polymers which modify the rheology of polar phases, and more particularly of aqueous phases, are mainly polyelectrolytes, resulting from the radical polymerization of (meth)acrylic type monomers, i.e. acrylic acid or methacrylic acid, esters derived from acrylic acid or methacrylic acid, or alternatively acrylamide or methacrylamide derivatives.

Developing new biobased and biodegradable rheology modifiers that are as efficient as the synthetic polymers currently used still constitutes a major challenge today. Indeed, until now the solutions mainly used for thickening aqueous phases involve ingredients originating from petrochemical materials and in particular acrylic acid and derivatives thereof, or methacrylic acid and derivatives thereof.

Given the growing concern of consumers for economy and sustainable and responsible development, replacing raw materials of petrochemical origin with raw materials of renewable origin to prepare polymers is a priority research area.

To date, the literature describes the use of various natural polymers or polymers from renewable raw materials, the monomer units of which come from the family of sugars (glucose, arabinose, xylose, galactose, mannose, ribose, glucuronic acid, etc.) or from the family of amino acids (glutamic acid, aspartic acid, lysine, etc.). These polymers are predominantly linear or branched depending on the plant from which they are obtained or depending on their manufacturing process.

As an example of a polymer of natural origin, mention may be made of polyglutamic acid (PGA), which nowadays is the subject of numerous research studies. It is a predominantly linear polymer and consists of glutamic acid (GA) monomer units. Glutamic acid is an amino acid characterized by an amine function in the α position and by two carboxylic acid functions (or carboxylates depending on the pH) in the α and γ positions (cf. chemical formula No. 1).

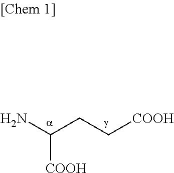

Chemical structure of glutamic acid (GA).

One of the ways to increase the branching of a synthetic or natural polymer or a polymer of natural origin consists in performing crosslinking reactions. The aim of the crosslinking of the polymer chains is to connect several polymer chains to one another which, when added to a polar phase, and more particularly to water, take the form of a three-dimensional network that is insoluble in water but is water-swellable, thus resulting in a chemical gel.

Crosslinked polymers can be prepared:
In one step by reacting the monomers and the crosslinking agent during the polymerization reaction, or
In at least two steps, of which the first consists in preparing the polymer and the second consists in reacting the polymer with a crosslinking agent to obtain a crosslinked polymer.

Various reactions exist for the crosslinking of polyglutamic acid (PGA), which makes it possible to obtain polymers of natural origin with improved thickening properties in polar media, and in particular in aqueous media. Among the crosslinking agents known to be used in the (PGA) crosslinking reaction, polyepoxide derivatives are the most widely described since they make it possible to perform crosslinking processes under environmentally friendly conditions (moderate temperature, reaction in aqueous media and in the absence of harmful solvents).

However, the implementation of these processes requires diluting the (PGA) to high levels, which leads to the production of an aqueous gel comprising, per 100% of its mass, a content by mass of less than or equal to 10% of a polymer (P), which is difficult for formulators to implement.

Proceeding from this, a problem that arises is that of providing an easy-to-use pharmaceutical composition for topical use comprising polymers of natural origin, the raw materials of which are renewable and which have thickening properties for polar media and more particularly for aqueous media.

One solution of the present invention is a pharmaceutical composition (F) for topical use comprising at least one pharmaceutical active principle and, as thickening agent, a polymer (P) in the form of a pulverulent solid consisting of monomer units derived from partially or totally salified glutamic acid (GA) and of monomer units of at least one crosslinking agent (XLA) hearing at least two glycidyl functions.

For the purposes of the invention, the term “pulverulent solid” denotes a solid consisting of fine particles that not bonded together or are bonded together only to a minor extent.

In the polymer (P) present in the pharmaceutical composition (F) for topical use that is the subject of the present invention, the monomer units derived from partially or totally salified glutamic acid (GA) are linked together:
either in such a way that the amine function of a glutamic acid (GA) monomer unit is covalently bonded with the carboxylic function located in the alpha (a) position of a second glutamic acid (GA) monomer unit; the resulting polymer is then called partially or totally salified “α-polyglutamic acid” or PAGA (cf. chemical formula No. 2),

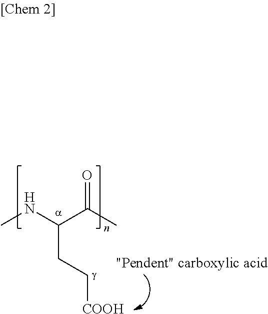

Chemical structure of α-polyglutamic acid or PAGA.
or in such a way that the amine function of a glutamic acid (GA) monomer unit is covalently bonded to the carboxylic function of the side chain located in the gamma (γ) position of a second glutamic acid (GA) monomer unit; the resulting polymer is then called partially or totally salified “γ-polyglutamic acid” or PGGA (cf. chemical formula No. 3).

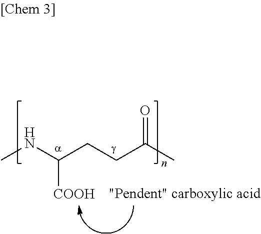

Chemical structure of γ-polyglutamic acid or PGGA.

In general, the PGA can be prepared chemically according to peptide synthesis methods known to those skilled in the art, in particular proceeding via steps of selective protection, activation, coupling and deprotection. The coupling generally consists of a nucleophilic attack of the amine function of one glutamic acid monomer unit on an activated carboxylic acid function of another glutamic acid monomer unit.

PGGA can also be obtained via processes comprising at least one microbial fermentation step involving the use of at least one bacterial strain.

For the purposes of the present invention, in the polymer (P) as defined above, the term “salified” indicates that the “pendent” carboxylic acid function present on each glutamic acid (GA) monomer unit of the polymer (in the gamma position in the case of PAGA or in the alpha position in the case of PGGA) is present in an anionic or carboxylate form. The counterion of this carboxylate function is a cation derived, for example, from salts of alkali metals such as sodium or potassium or salts of nitrogenous bases such as amines, lysine or monoethanolamine (HO—CH₂—CH₁—NH₂).

For the purposes of the present invention, the term “crosslinking agent (XLA) bearing at least two glycidyl functions” denotes a crosslinking agent (XLA) as defined above, the molecular structure of which comprises at least two glycidyl units or functions of formula (I′):

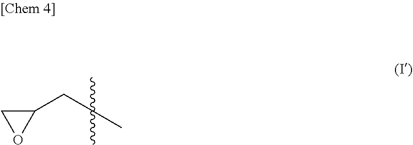 [SMILES: *CC1CO1]

The crosslinking of the polymer chains of the polymer (P) is performed according to a reaction between the terminal free amine function (—NH2) and/or one or more “pendent” or terminal carboxylic or carboxylate functions (—COOH or —COO⁻ present in the structure of said polymer (P), and at least one epoxy group present in the structure of the crosslinking agent (XLA) bearing at least two glycidyl functions.

For the purposes of the present invention, the term “pharmaceutical active principle” denotes a chemical substance which is included in the composition of a medicament and which exhibits a therapeutic or prophylactic effect.

The crosslinking agent (XLA) may be chosen from the members of the group consisting of:
monoethylene glycol diglycidyl ether of formula (I):

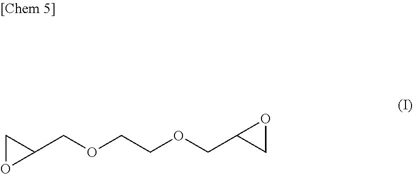 [SMILES: C(COCC1CO1)OCC1CO1]

the compound of formula (II):

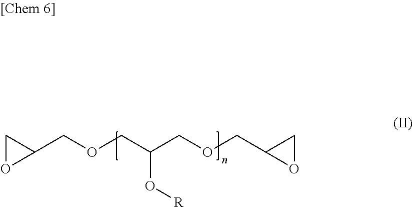

with R representing a hydrogen atom or the glycidyl radical

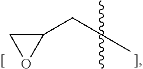 [SMILES: *CC1CO1]

and n representing an integer greater than or equal to 1 and less than or equal to 10;
1,3-propanediol diglycidyl ether of formula (III):

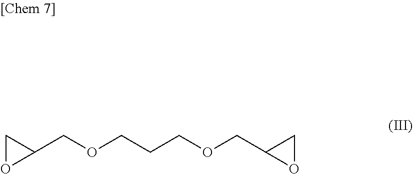 [SMILES: C(COCC1CO1)COCC1CO1]

1,2-propanediol diglycidyl ether of formula (IV):

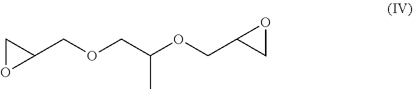

1,4-butanediol diglycidyl ether of formula (V):

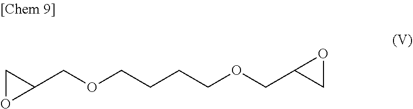 [SMILES: C(CCOCC1CO1)COCC1CO1]

1,2-butanediol diglycidyl ether of formula (VI):

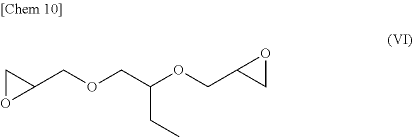

1,3-butanediol diglycidyl ether of formula (VII):

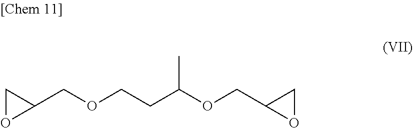

1,6-hexanediol diglycidyl ether of formula (VIII):

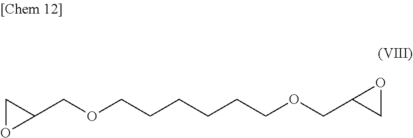 [SMILES: C(CCCOCC1CO1)CCOCC1CO1]

the compound of formula (IX):

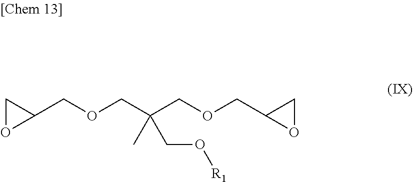

with R1 representing a hydrogen atom or the glycidyl radical

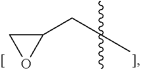 [SMILES: *CC1CO1]

the compound of formula (X):

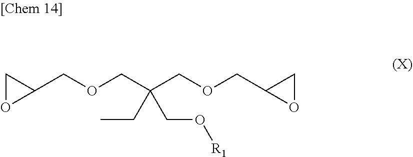

with R1 representing a hydrogen atom or the glycidyl radical

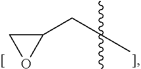 [SMILES: *CC1CO1]

the compound of formula (XI):

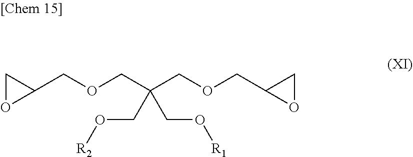

with R1 and R2 independently representing a hydrogen atom or the glycidyl radical

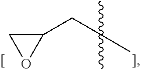 [SMILES: *CC1CO1]

the compound of formula (XII):

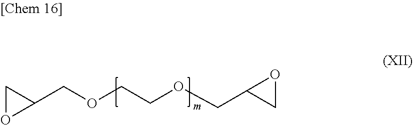

with m representing an integer greater than or equal to 2,
the compound of formula (XIII):

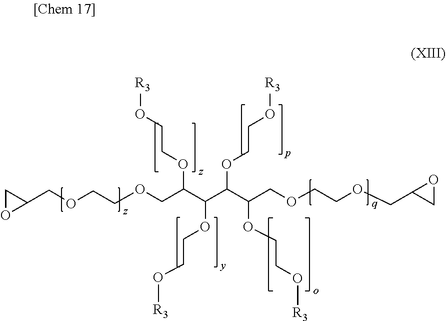

with R3 representing a hydrogen atom or the glycidyl radical

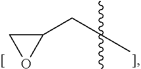 [SMILES: *CC1CO1]

and x, y, z, o, p and q, independently of one another, representing an integer greater than or equal to 2 and less than or equal to 10.

Depending on the case, the pharmaceutical composition for topical use (F) may have one or more of the following characteristics:
in the polymer (P), per 100 mol % of monomer units derived from partially or totally salified glutamic acid (GA), the crosslinking agent (XLA) represents from 0.5 mol % to 20 mol %, more particularly from 0.5 mol % to 15 mol %, and more particularly still from 0.5 mol % to 12 mol %.
the polymer (P) additionally comprises a compound of formula (XIV):

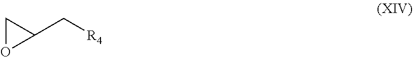

with R4 representing a linear or branched, saturated or unsaturated, hydrocarbon-based radical, optionally containing at least one hydroxyl function, being functionalized or non-functionalized and containing from 6 to 22 carbon atoms.
in formula (XIV), R4 represents a hydrocarbon-based radical chosen from the elements of the group consisting of the heptyl, octyl, nonyl, decyl, undecyl, undecenyl, dodecyl, tridecyl, tetradecyl, pentadecyl, hexadecyl, heptadecyl, octadecyl, hydroxyoctadecyl, oleyl, linoleyl, linolenyl, eicosyl and dodecosyl radicals.
in formula (XIV), R4 represents a hydrocarbon-based radical chosen from the elements of the group consisting of the n-octyl, n-nonyl, n-decyl, n-undecyl, n-undecenyl, n-dodecyl, n-tetradecyl, n-hexadecyl, n-octadecyl, 12-hydroxyoctadecyl, n-eicosyl and n-dodecosyl radicals.
in said polymer (P), per 100% of the mass of monomer units derived from partially or totally salified glutamic acid (GA), the compound of formula (XIV) represents from 1% to 50% by mass.

PGGA can exist in various conformational forms in solution in water. These forms depend on the inter- and intramolecular hydrogen bonds and thus on the pH, the polymer concentration, the ionic strength of the solution, and also the temperature. The PGGA chains can thus adopt an a helix form, a 13 sheet form, aggregate form or else be in a disordered and random state.

According to one particular aspect, the polymer (P) is in helical conformation when it is present in a solution at a content by mass of less than or equal to 0.1% and said aqueous solution of which exhibits a pH of less than or equal to 7.

According to one particular aspect, the polymer (P) is in sheet conformation when it is present in an aqueous solution at a content by mass of less than or equal to 0.1% and said aqueous solution of which exhibits a pH of greater than 7.
The pharmaceutical composition for topical use (F) comprises, per 100% of its mass, between 0.1% and 10% by mass of said polymer (P), more particularly between 0.1% and 8% by mass, and more particularly still between 0.1% and 5% by mass.
The pharmaceutical active principle is chosen from antibacterial agents, antimicrobial agents, antiparasitic agents, antihelminthic agents, anticoccidial agents, anti-Cryptosporidium agents, anti-protozoal agents, antimycotic agents, non-steroidal anti-inflammatory agents, antiallergic and immunomodulatory agents, analgesic agents, antihistaminic agents, local anesthetic agents, insecticidal agents, antiseptic agents and antifungal agents.

By way of example:
said pharmaceutical composition (F) will comprise as pharmaceutical active principle a nonsteroidal anti-inflammatory agent and said pharmaceutical composition (F) will be for use in reducing and/or eliminating local pains, post-traumatic inflammation of the joints, muscles, tendons or ligaments, localized forms of soft-tissue rheumatism, localized forms of degenerative joint disease, actinic keratosis caused by overexposure to sunlight, acute migraine, pain associated with bone metastases, fever due to malignant lymphogranulomatosis (Hodgkin's lymphoma), multi-drug resistant E. coli, Shy-Drager syndrome and diabetes mellitus.
said pharmaceutical composition (F) will comprise as pharmaceutical active principle a local anesthetic and said pharmaceutical composition (F) will be for use in treating pain, pruritus and/or anorectal disorders in humans or animals.
said pharmaceutical composition (F) will comprise as pharmaceutical active principle an antifungal agent and said pharmaceutical composition (F) will be for use in treating mycoses of the skin, scalp, mouth and/or gynecological apparatus in human or animal mammals.

The present invention also provides a process for preparing a polymer (P) as defined above, comprising:
a step A) of preparing a polymer (P), comprising the following substeps:
   a. a step of preparing a polar phase (PP) comprising partially or totally salified PGA, at least one polar solvent (PS) and at least one crosslinking agent (XLA) comprising at least two glycidyl functions,
   b. a step of adjusting the pH of the aqueous solution obtained in step a) to a pH of between 3 and 11, and
   c. a step of spray-drying the polar phase (PP) resulting from step b) so as to obtain the polymer (P) and
a step B) of mixing at least one polymer (P) prepared during step A) with at least one pharmaceutical active principle.

Depending on the case, the process according to the invention may have one or more of the following characteristics:

In Step a):
The polyglutamic acid involved is gamma-polyglutamic acid (PGGA),
All of the monomer units constituting the PGA are derived from sodium glutamate, potassium glutamate, ammonium glutamate, calcium glutamate, magnesium glutamate or a mixture of these forms,
The polar phase (PP) comprises, per 100% of its mass: from 5% to 80% by mass of the polyglutamic acid (PGA) of the polymer (P), from 0.025% to 8% by mass of the crosslinking agent (XLA), from 12% to 94.975% by mass of at least one polar solvent,
The polar solvent is chosen from the elements of the group consisting of water, methanol, ethanol, 1-propanol, 2-propanol, isobutanol, tert-butanol, 2-methyl-2-propanol, 1-butanol, 2-butanol, acetone, dimethyl ketone, diethyl ketone, tetrahydropyran, tetrahydrofuran, 2-methyltetrahydrofuran, 1,3-dioxane and 1,4-dioxane. The polar phase (PP) additionally comprises at least one compound of formula (XIV):

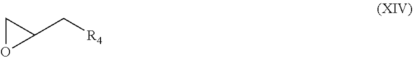

with R4 representing a linear or branched, saturated or unsaturated hydrocarbon-based radical, optionally containing at least one hydroxyl function and containing from 6 to 22 carbon atoms, R4 representing a hydrocarbon-based radical chosen from the elements of the group consisting of the heptyl, octyl, nonyl, decyl, undecyl, undecenyl, dodecyl, tridecyl, tetradecyl, pentadecyl, hexadecyl, heptadecyl, octadecyl, hydroxyoctadecyl, oleyl, linoleyl, linolenyl, eicosyl and dodecosyl radicals.

According to one particular aspect, in formula (XIV), R4 represents a hydrocarbon-based radical chosen from the elements of the group consisting of the n-octyl, n-nonyl, n-decyl, n-undecyl, n-undecenyl, n-dodecyl, n-tetradecyl, n-hexadecyl, n-octadecyl, 12-hydroxyoctadecyl, n-eicosyl and n-dodecosyl radicals.
The content of compound of formula (XIV) in the polar phase (PP) is, per 100% by mass of said polar phase (PP), between 0.05% and 35% by mass, it being understood that the sum of the proportions by mass of the polyglutamic acid (PGA), of the crosslinking agent (XLA), of the polar solvent (PS) and of the compound of formula (XIV) is equal to 100%.

In Step b):
The pH is between 4 and 10, preferably between 5 and 7.

In Step c):
The drying is carried out using an atomizer employing an air flow and a nozzle,
The inlet temperature of the air flow is between 80° C. and 180° C., preferably between 100° C. and 170° C.

According to one particular aspect, the polar phase (PP) comprises, per 100% of its mass: from 5% to 70% by mass of the polyglutamic acid (PGA) of the polymer (P), from 0.025% to 7% by mass of the crosslinking agent (XLA), from 23% to 94.975% by mass of at least one polar solvent (PS).

According to an even more particular aspect, the polar phase (PP) comprises, per 100% of its mass: from 5% to 60% by mass of the polyglutamic acid (PGA) of the polymer (P), from 0.025% to 6% by mass of the crosslinking agent (XLA), from 34% to 94.975% by mass of at least one polar solvent (PS).

According to another particular aspect, the polar solvent (PS) is chosen from the following list: water, ethanol, acetone, dimethyl ketone.

According to another even more particular aspect, the polar solvent (PS) is water.

According to another particular aspect, R4, representing a hydrocarbon-based radical, is chosen from the elements of the group consisting of the n-octyl, n-nonyl, n-decyl, n-undecyl, n-undecenyl, n-dodecyl, n-tetradecyl, n-hexadecyl, n-octadecyl, 12-hydroxyoctadecyl, n-eicosyl and n-dodecosyl radicals.

According to another particular aspect, the content of compound of formula (XIV) in the polar phase (PP) is, per 100% by mass of said polar phase (PP), between 0.05% and 20% by mass, and more particularly still between 0.05% and 15% by mass, it being understood that the sum of the proportions by mass of the polyglutamic acid (PGA), of the crosslinking agent (XLA), of the polar solvent (PS) and of the compound of formula (XIV) is equal to 100%.

The invention also provides for the use of said polymer (P) as defined above, as thickening and/or emulsifying and/or stabilizing agent for a pharmaceutical composition (F) for topical use.

According to one particular aspect, said use consists in thickening polar phases, for instance aqueous, alcoholic or aqueous-alcoholic phases or polar phases comprising polyols such as glycerol.

According to another particular aspect, said use consists in stabilizing an emulsion of oil-in-water type, or of water-in-oil type, giving said emulsion a homogeneous appearance during storage under various conditions, and more particularly at 25° C. for a time at least equal to one month, and more particularly at 4° C. for a time at least equal to one month, and more particularly at 45° C. for a time at least equal to one month.

According to another particular aspect, said use consists in stabilizing solid particles in pharmaceutical compositions (F) for topical use.

These solid particles to be suspended may have various regular or irregular geometries, and may be in the form of pearls, beads, rods, flakes, leaflets or polyhedra. These solid particles are characterized by an apparent mean diameter of between 1 micrometer and 5 millimeters, more particularly between 10 micrometers and 1 millimeter.

The solid particles that may be suspended and stabilized with the polymer (P) as defined previously in pharmaceutical compositions for topical use include micas, iron oxide, titanium oxide, zinc oxide, aluminum oxide, talc, silica, kaolin, clays, boron nitride, calcium carbonate, magnesium carbonate, magnesium hydrogencarbonate, inorganic colored pigments, polyamides, such as nylon-6, polyethylenes, polypropylenes, polystyrenes, polyesters, acrylic or methacrylic polymers, such as polymethyl methacrylates, polytetrafluoroethylene, crystalline or microcrystalline waxes, porous spheres, selenium sulfide, zinc pyrithione, starches, alginates, plant fibers, loofah particles and sponge particles.

Said pharmaceutical composition (F) for topical use, which is a subject of the present invention, is in particular in the form of an aqueous solution, an emulsion or a microemulsion with an aqueous continuous phase, an emulsion or a microemulsion with an oily continuous phase, an aqueous gel, a foam, or in the form of an aerosol. It may be applied directly to the surface of the skin or else via any type of support intended to be placed in contact with the surface of the skin (paper, wipe, textile).

In general, said pharmaceutical composition for topical use (F) which is a subject of the present invention also includes at least one or more auxiliary compounds chosen from fatty phases, foaming and/or detergent surfactants, thickening and/or gelling surfactants, thickening and/or gelling agents, stabilizers, film-forming compounds, solvents and cosolvents, hydrotropic agents, plasticizers, opacifiers, pearlescent agents, superfatting agents, sequestrants, chelating agents, antioxidants, fragrances, essential oils, preserving agents, conditioning agents and deodorants.

In general, the pharmaceutical for topical use (F) according to the invention may comprise excipients and/or active principles usually used in the field of formulations for topical use, in particular pharmaceutical or dermopharmaceutical formulations.

Among the fatty phases present in the pharmaceutical composition (F) for topical use according to the invention, mention may be made of oils and waxes.

The term “oil” denotes a compound and/or a mixture of compounds that are water-insoluble, and liquid at 25° C., and more particularly:
linear alkanes comprising from 11 to 19 carbon atoms;
branched alkanes comprising from 7 to 40 carbon atoms, such as isododecane, isopentadecane, isohexadecane, isoheptadecane, isooctadecane, isononadecane or isoeicosane, or mixtures of some thereof such as those mentioned below and identified by their INCI name: C₇₋₈ isoparaffin, C₈₋₉ isoparaffin, C₉₋₁₁ isoparaffin, C₉₋₁₂ isoparaffin, C₉₋₁₃ isoparaffin, C₉-14 isoparaffin, C₉₋₁₆ isoparaffin, isoparaffin, C₁₀₋₁₂ isoparaffin, C₁₀₋₁₃ isoparaffin, C₁₁₋₁₂ isoparaffin, C₁₁₋₁₃ isoparaffin, C₁₁₋₁₄ isoparaffin, C₁₂₋₁₄ isoparaffin, C₁₂₋₂₀ isoparaffin, C₁₃₋₁₄ isoparaffin, C₁₃₋₁₆ isoparaffin;
cycloalkanes optionally substituted by one or more linear or branched alkyl radicals;
white mineral oils, such as those sold under the following names: Marcol™ 52, Marcol™ 82, Drakeol™ 6VR, Eolane™ 130, Eolane™ 150;
hemisqualane (or 2,6,10-trimethyldodecane; CAS number: 3891-98-3), squalane (or 2,6,10,15,19,23-hexamethyltetracosane), hydrogenated polyisobutene or hydrogenated polydecene;
mixtures of alkanes comprising from 15 to 19 carbon atoms, said alkanes being linear alkanes, branched alkanes and cycloalkanes, and more particularly the mixture (MI) which comprises, per 100% of its mass, a proportion by mass of branched alkanes of greater than or equal to 90% and less than or equal to 100%; a proportion by mass of linear alkanes of greater than or equal to 0% and less than or equal to 9%, and more particularly less than 5%, and a proportion by mass of cycloalkanes of greater than or equal to 0% and less than or equal to 1%, for example the mixtures sold under the name Emogreen™ L15 or Emogreen™ L19;
fatty alcohol ethers of formula (II′):

Z₁—O—Z₂ (II′),

in which Z₁ and Z₂, which may be identical or different, represent a linear or branched alkyl radical comprising from 5 to 18 carbon atoms, for example dioctyl ether, didecyl ether, didodecyl ether, dodecyl octyl ether, dihexadecyl ether, (1,3-dimethylbutyl) tetradecyl ether, (1,3-dimethylbutyl) hexadecyl ether, bis(1,3-dimethylbutyl) ether or dihexyl ether;
monoesters of fatty acids and of alcohols of formula (III′):

R′₁—(C═O)—O—R′₂ (III′),

in which R′₁—(C═O) represents a saturated or unsaturated, linear or branched acyl radical comprising from 8 to 24 carbon atoms, and R′2 represents, independently of R′₁, a saturated or unsaturated, linear or branched hydrocarbon-based chain comprising from 1 to 24 carbon atoms, for example methyl laurate, ethyl laurate, propyl laurate, isopropyl laurate, butyl laurate, 2-butyl laurate, hexyl laurate, methyl cocoate, ethyl cocoate, propyl cocoate, isopropyl cocoate, butyl cocoate, 2-butyl cocoate, hexyl cocoate, methyl myristate, ethyl myristate, propyl myristate, isopropyl myristate, butyl myristate, 2-butyl myristate, hexyl myristate, octyl myristate, methyl palmitate, ethyl palmitate, propyl palmitate, isopropyl palmitate, butyl palmitate, 2-butyl palmitate, hexyl palmitate, octyl palmitate, methyl oleate, ethyl oleate, propyl oleate, isopropyl oleate, butyl oleate, 2-butyl oleate, hexyl oleate, octyl oleate, methyl stearate, ethyl stearate, propyl stearate, isopropyl stearate, butyl stearate, 2-butyl stearate, hexyl stearate, octyl stearate, methyl isostearate, ethyl isostearate, propyl isostearate, isopropyl isostearate, butyl isostearate, 2-butyl isostearate, hexyl isostearate, isostearyl isostearate;
diesters of fatty acids and of glycerol of formula (IV′) and of formula (V′):

R′₃—(C═O)—O—CH₂—CH(OH)—CH₂—O—(C═O)—R′4 (IV′)

R′₅—(C═O)—O—CH₂—CH┌O—(C═O)—R′₆┐—CH₂—OH (V′),

formulae (IV′) and (V′) in which R′₃—(C═O), R′₄—(C═O), R′₅—(C═O) and R′₆—(C═O), which may be identical or different, represent a saturated or unsaturated, linear or branched acyl group comprising from 8 to 24 carbon atoms;
triesters of fatty acids and of glycerol of formula (VI′):

R′₇—(C═O)—O—CH₂—CH└O—(C═O)—R″₈┘—CH₂—O—(C═O)—R″₉ (NT),

in which R′₇—(C═O), R′₈—(C═O) and R′₉—(C═O), which may be identical or different, represent a saturated or unsaturated, linear or branched acyl group comprising from 8 to 24 carbon atoms;
plant oils, such as phytosqualane, sweet almond oil, coconut oil, castor oil, jojoba oil, olive oil, rapeseed oil, peanut oil, sunflower oil, wheat germ oil, corn germ oil, soybean oil, cottonseed oil, alfalfa oil, poppy oil, red kuri squash oil, evening primrose oil, millet oil, barley oil, rye oil, safflower oil, candlenut oil, passionflower oil, hazelnut oil, palm oil, shea butter, apricot kernel oil, calophyllum oil, sisymbrium oil, avocado oil, calendula oil, and oils obtained from flowers or vegetables;
ethoxylated plant oils.

Preferably, the composition according to the invention comprises at least one oil chosen from the elements of the group consisting of castor oil, liquid paraffins, cocoyl caprylate/caprate, isopropyl myristate and capric/caprylic triglyceride.

Among the fatty phases which can be combined with the pharmaceutical composition (F) for topical use according to the invention, mention may be made of waxes. These waxes are more particularly chosen from beeswax, carnauba wax, candelilla wax, ouricury wax, Japan wax, cork fiber wax, sugarcane wax, paraffin waxes, lignite waxes, microcrystalline waxes, lanolin wax; ozokerite; polyethylene wax; silicone waxes; plant waxes; fatty alcohols and fatty acids which are solid at ambient temperature; glycerides which are solid at ambient temperature.

As regards the auxiliary compounds, among the foaming and/or detergent anionic surfactants which can be combined with the pharmaceutical composition (F) for topical use according to the invention, mention may be made of alkali metal salts, alkaline-earth metal salts, ammonium salts, amine salts or amino alcohol salts of alkyl ether sulfates, of alkyl sulfates, of alkylamido ether sulfates, of alkylaryl polyether sulfates, of monoglyceride sulfates, of alpha-olefin sulfonates, of paraffin sulfonates, of alkyl phosphates, of alkyl ether phosphates, of alkyl sulfonates, of alkylamide sulfonates, of alkylaryl sulfonates, of alkyl carboxylates, of alkyl sulfosuccinates, of alkyl ether sulfosuccinates, of alkylamide sulfosuccinates, of alkyl sulfoacetates, of alkyl sarcosinates, of acyl isethionates, of N-acyl taurates, of acyl lactylates, of N-acylated derivatives of amino acids, of N-acylated derivatives of peptides, of N-acylated derivatives of proteins, or of fatty acids.

Among the foaming and/or detergent amphoteric surfactants optionally present in the pharmaceutical composition (F) for topical use according to the invention, mention may be made of alkylbetaines, alkylamidobetaines, sultaines, alkylamidoalkylsulfobetaines, imidazoline derivatives, phosphobetaines, amphopolyacetates and amphopropionates.

Among the foaming and/or detergent cationic surfactants optionally present in the pharmaceutical composition (F) for topical use according to the invention, mention may be made in particular of quaternary ammonium derivatives.

Among the foaming and/or detergent nonionic surfactants optionally present in the composition (F) for topical use according to the invention, mention may be made more particularly of alkyl polyglycosides comprising a linear or branched, saturated or unsaturated aliphatic radical and comprising from 8 to 12 carbon atoms; castor oil derivatives, polysorbates, coconut amides and N-alkylamines.

As examples of thickening and/or gelling surfactants optionally present in the pharmaceutical composition (F) for topical use according to the invention, mention may be made of:
optionally alkoxylated fatty esters of alkyl polyglycosides, and most particularly ethoxylated esters of methyl polyglucoside such as PEG 120 methyl glucose trioleate and PEG 120 methyl glucose dioleate sold, respectively, under the names Glucamate™ LT and Glumate™ DOE120;
alkoxylated fatty esters, such as PEG 150 pentaerythrityl tetrastearate, sold under the name Crothix™ DS53, or PEG 55 propylene glycol oleate, sold under the name Antil™ 141;
carbamates of polyalkylene glycols comprising fatty chains, such as PPG 14 laureth isophoryl dicarbamate, sold under the name Elfacos™ T211, or PPG 14 palmeth 60 hexyl dicarbamate, sold under the name Elfacos™ GT2125.

As examples of emulsifying surfactants optionally present in the pharmaceutical composition (F) for topical use according to the invention, mention may be made of nonionic surfactants, anionic surfactants and cationic surfactants.

As examples of emulsifying nonionic surfactants optionally present in the pharmaceutical composition (F) for topical use according to the invention, mention may be made of ethoxylated castor oil and ethoxylated hydrogenated castor oil, for example the product sold under the name Simulsol™ 989; compositions comprising glycerol stearate and stearic acid poly(ethoxylated) with between 5 mol and 150 mol of ethylene oxide, for example the composition comprising stearic acid (ethoxylated) with 135 mol of ethylene oxide and glycerol stearate sold under the name Simulsol™ 165; ethoxylated sorbitan esters, for example the products sold under the name Montanox™; ethoxylated mannitan esters; sucrose esters; methyl glucoside esters.

As examples of emulsifying anionic surfactants optionally present in the pharmaceutical composition (F) for topical use according to the invention, mention may be made of decyl phosphate, cetyl phosphate sold under the name Amphisol™, glyceryl stearate citrate; cetearyl sulfate; the arachidyl/behenyl phosphates and arachidyl/behenyl alcohols composition sold under the name Sensanov™ WR; soaps, for example sodium stearate or triethanolammonium stearate, or N-acylated derivatives of amino acids which are salified, for instance stearoyl glutamate.

As examples of emulsifying cationic surfactants optionally present in the pharmaceutical composition (F) for topical use according to the invention, mention may be made of amine oxides, quaternium-82, cetyltrimethyl ammonium chloride, hexadecyltrimethylammonium bromide, cetylpyridinium chloride, benzalkonium chloride, benzethonium chloride, and the surfactants described in the document WO 96/00719 and mainly those in which the fatty chain comprises at least 16 carbon atoms.

As examples of opacifiers and/or pearlescent agents optionally present in the pharmaceutical composition (F) for topical use according to the invention, mention may be made of sodium palmitate, sodium stearate, sodium hydroxystearate, magnesium palmitate, magnesium stearate, magnesium hydroxystearate, ethylene glycol monostearate, ethylene glycol distearate, polyethylene glycol monostearate, polyethylene glycol distearate, and fatty alcohols comprising from 12 to 22 carbon atoms.

As examples of texturing agents optionally present in the pharmaceutical composition (F) for topical use according to the invention, mention may be made of N-acylated derivatives of amino acids, for example lauroyl lysine sold under the name Aminohope™ LL, octenyl starch succinate sold under the name Dryflo™, myristyl polyglucoside sold under the name Montanov 14, cellulose fibers, cotton fibers, chitosan fibers, talc, sericite and mica.

As examples of solvents and cosolvents optionally present in the pharmaceutical composition (F) for topical use according to the invention, mention may be made of water, organic solvents, for example glycerol, diglycerol, glycerol oligomers, ethylene glycol, propylene glycol, butylene glycol, hexylene glycol, diethylene glycol, xylitol, erythritol, sorbitol, water-soluble alcohols such as ethanol, isopropanol or butanol, mixtures of water and of said organic solvents, propylene carbonate, ethyl acetate, benzyl alcohol and dimethyl sulfoxide (DMSO).

As examples of agents for improving the skin penetration that are optionally present in the pharmaceutical composition (F) for topical use according to the invention, mention may be made of glycol ethers, for instance ethylene glycol monomethyl ether, ethylene glycol monoethyl ether, ethylene glycol monopropyl ether, ethylene glycol monoisopropyl ether, ethylene glycol monobutyl ether, ethylene glycol monophenyl ether, ethylene glycol monohenzyl ether, diethylene glycol monomethyl ether, diethylene glycol monoethyl ether and diethylene glycol mono(n-butyl) ether, diethylene glycol monoethyl ether (or Transcutol-P), fatty acids such as oleic acid, fatty acid esters of glycerol, for instance glyceryl behenate, glyceryl palmitostearate, behenoyl macroglycerides, polyoxyethylene (2) stearyl ether, polyoxyethylene (2) oleyl ether, terpenes, for instance D-limonene, and essential oils, for instance the essential oil of Eucalyptus.

As examples of thickening and/or gelling agents optionally present in the pharmaceutical composition (F) for topical use according to the invention, mention may be made of polysaccharides consisting only of monosaccharides, such as glucans or glucose homopolymers, glucomannoglucans, xyloglycans, galactomannans of which the degree of substitution (DS) of the D-galactose units on the main D-mannose chain is between 0 and 1, and more particularly between 1 and 0.25, such as galactomannans originating from cassia gum (DS=1/5), locust bean gum (DS=1/4), tara gum (DS=1/3), guar gum (DS=1/2) or fenugreek gum (DS=1).

As examples of thickening and/or gelling agents optionally present in the pharmaceutical composition (F) for topical use according to the invention, mention may be made of polysaccharides consisting of monosaccharide derivatives, such as sulfated galactans and more particularly carrageenans and agar, uronans and more particularly algins, alginates and pectins, heteropolymers of monosaccharides and uronic acids, and more particularly xanthan gum, gellan gum, gum arabic exudates and karaya gum exudates, and glucosaminoglycans.

As examples of thickening and/or gelling agents optionally present in the pharmaceutical composition (F) for topical use according to the invention, mention may be made of cellulose, cellulose derivatives such as methylcellulose, ethylcellulose, hydroxypropylcellulose, silicates, starch, hydrophilic starch derivatives, and polyurethanes.

As examples of stabilizers optionally present in the pharmaceutical composition (F) for topical use according to the invention, mention may be made of microcrystalline waxes, and more particularly ozokerite, and mineral salts such as sodium chloride or magnesium chloride.

As examples of thermal or mineral waters which can be combined with the pharmaceutical composition (F) for topical use according to the invention, mention may be made of thermal or mineral waters having a mineralization of at least 300 mg/l, in particular Avene water, Vittel water, Vichy basin water, Uriage water, La Roche-Posay water, La Bourboule water, Enghien-les-Bains water, Saint-Gervais-les-Bains water, Néris-les-Bains water, Allevard-les-Bains water, Digne water, Maizières water, Neyrac-les-Bains water, Lons-le-Saunier water, Rochefort water, Saint Christau water, Les Fumades water and Tercis-les-Bains water.

As examples of active agents which can be combined with the pharmaceutical composition (F) for topical use according to the invention, mention may be made of substances or compositions which provide a beneficial effect to the human or animal subject.

These active agents may, for example, be antibodies, analgesics, anti-inflammatories, cytokines, cytoxins, growth factors, hormones, lipids, oligonucleotides, polymers, polysaccharides, polypeptides, protease inhibitors, vitamins, insect repellents, antibiotics or anti-inflammatory agents.

As examples of analgesic and anti-inflammatory agents which can be combined with the pharmaceutical composition (F) for topical use according to the invention, mention may be made of acetaminophen, aspirin, salicylic acid, methyl salicylate, choline salicylate, glycol salicylate, 1-menthol, camphor, mefenamic acid, fluphenamic acid, indomethacin, protizidic acid, fentiazac, tolmetin, tiaprofenic acid, phenylbutazone, oxyphenbutazone, clofezone, pentazocine, mepirizole, hydrocortisone, cortisone, dexamethasone, fluocinolone, triamcinolone, medrysone, prednisolone, flurandrenolide, prednisone, halcinonide, methylprednisolone, fludrocortisone, corticosterone, paramethasone and betamethasone.

As examples of non-steroidal anti-inflammatory agents (or NSAIDs) which can be combined with the pharmaceutical composition (F) for topical use according to the invention, mention may be made more particularly of arylacetic (or arylalkanoic) derivatives and 2-arylpropionic acids (or profens), and even more particularly diclofenac, tiaprofenic acid, alminoprofen, etodolac, flurbiprofen, ibuprofen, ketoprofen and naproxen.

As examples of antiseptic agents which can be combined with the pharmaceutical composition (F) for topical use according to the invention, mention may be made of cetrimide, povidone-iodine, chlorhexidine, iodine, benzalkonium chloride, benzoic acid, nitrofurazone, benzoyl peroxide, hydrogen peroxide, hexachlorophene, phenol, resorcinol and cetylpyridinium chloride.

As examples of insecticidal agents which can be combined with the pharmaceutical composition (F) for topical use according to the invention, mention may be made of trichlorfon, triflumuron, fenthion, bendiocarb, cyromazine, diflubenzuron, dicyclanil, fluazuron, amitraz, deltamethrin, cypermethrin, chlorfenvinphos, flumethrin, ivermectin, abamectin, avermectin, doramectin, moxidectin, zeta-cypermethrin, diazinon, spinosad, imidacloprid, nitenpyram, pyriproxyfen, fipronil, cythioate, lufenuron, selamectin, milbemycin oxime, chlorpyrifos, coumaphos, propetamphos, alpha-cypermethrin, cypermethrin high cis, ivermectin, diflubenzuron, cyclodiene, carbamate and benzoylurea.

As examples of antimicrobial agents which can be combined with the pharmaceutical composition (F) for topical use according to the invention, mention may be made of sulfonamides, aminoglycosides, for instance neomycin, tobramycin, gentamicin, amikacin, kanamycin, spectinomycin, paromomycin, netilmicin, polypeptides, cephalosporins, oxazolidinones, for instance ciprofloxacin, levofloxacin, and ofloxacin.

As examples of active agents which can be combined with the pharmaceutical composition (F) for topical use according to the invention, mention may be made of vitamin E, Coenzyme Q10, L-carnitine, choline, folic acid, magnesium and salts thereof, caprylic acid, linoleic acid, lauric acid, taurine, vitamin C, vitamin A, and vitamins of the B group.

### EXAMPLES

The examples that follow illustrate the invention without, however, limiting it.

### Example 1: Preparation of a Sodium PGGA According to the Invention Crosslinked with Polyethylene Glycol Diglycidyl Ether and Atomized

The synthesis process comprises the following 4 steps:

Step 1): Production of an Aqueous Gel of Sodium PGGA:

200 grams of demineralized water are placed under stirring with a Rayneri™ brand mechanical stirrer equipped with a deflocculator-type rotor and 20 grams of PGGA, sold by Lubon under the name “cosmetic grade PGGA”, are then added slowly to the vortex.

Step 2): Adjustment of the pH:

The pH of the mixture from step 1) is adjusted to a value of between 5.5 and 6.0 using an aqueous 5 M solution of HCl (temperature=20° C.).

Step 3): Addition of Crosslinker:

To the mixture from step 2) are added 1.2 grams of polyethylene glycol diglycidyl ether (PEGDGE) with an average molecular weight of the order of 500 g/mol.

Step 4): Atomization:

The aqueous solution obtained in step 3) is concentrated by passage through an atomizer the operating parameters of which are as follows:
Rate of introduction of the solution=4 ml/min,
Pressure of spraying nozzle=1.5 bar,
Circulating air temperature=150° C.

Composition (E1) is lastly isolated in the form of a pulverulent powder.

### Example 2: Preparation of a Sodium PGGA According to the Invention Crosslinked with 1,4-Butanediol Diglycidyl Ether and Atomized

The synthesis process of example 1 is reproduced, replacing the 1.2 grams of polyethylene glycol diglycidyl ether (PEGDGE) with 0.48 grams of 1,4-butanediol diglycidyl ether sold under the name Erisys™ GE 21 by Emerald.

The composition (E2) obtained is lastly isolated in the form of a pulverulent powder.

Evaluation of the Crosslinked and Atomized Sodium PGGAs Obtained in the Two Preceding Examples:

The evaluation of compositions (E1) and (E2) (crosslinked and atomized sodium PGGAs), obtained in the two preceding examples, was carried out as follows:
In a 400 ml tall-form beaker, 4 grams of the polymer compositions to be tested in the form of a pulverulent powder are dispersed in 196 grams of water with stirring using a Rayneri™ brand mechanical stirrer equipped with a deflocculator-type rotor, until a homogeneous gel is obtained.
The dynamic viscosity is measured using a Brookfield RVT type viscometer, speed 5, choosing the appropriate spindle.
0.1% of NaCl is added to the gel previously produced and stirring is effected with a Rayneri™ brand mechanical stirrer equipped with a deflocculator-type rotor, until the mixture is homogenized.
The dynamic viscosity is measured using the same viscometer as above, always choosing the appropriate spindle.

A control was produced from noncrosslinked PGGA sold by Lubon under the name “cosmetic grade PGGA”.

The measured dynamic viscosity values are collated in the table below:

TABLE 1

| | Nature | Viscosity in | Viscosity in mPa · s of |
|---|---|---|---|
| | of the | mPa · s of the | the 2% polymer + 0.1% |
| Composition | crosslinker | 2% polymer gel | NaCl gel |
| Control^{a)} | No | 176 (spindle 2) | 128 (spindle 2) |
| Composition | PEGDGE | 30 400 (spindle 5) | 9320 (spindle 3) |
| (E1) | | | |
| Composition | Erisys ™ | 41 520 (spindle 5) | / |
| (E2) | GE 21 | | |

^{a)}Non-crosslinked PGGA sold by Lubon under the name “cosmetic grade PGGA”.

Dynamic viscosities of aqueous gels obtained with compositions (E1) and (E2).

The control sodium PGGA gels (non-crosslinked and non-atomized) exhibit viscosities of between 100 and 200 mPa·s in the presence or absence of NaCl.

In the absence of NaCl, crosslinked and atomized sodium PGGA gels have viscosities respectively equal to 30 400 mPa·s in the case where the crosslinker is PEGDGE and 41 520 mPa·s with Erisys™ GE 21 as crosslinker.

In the presence of NaCl, the viscosity of the gel of sodium PGGA crosslinked by PEGDGE and atomized is equal to 9320 mPa·s.

Compositions (E1) and (E2) according to the invention therefore make it possible to thicken aqueous phases.

1. A pharmaceutical composition (F) for topical use comprising at least one pharmaceutical active principle and, as thickening agent, a polymer (P) in the form of a pulverulent solid consisting of monomer units derived from partially or totally salified glutamic acid (GA) and of monomer units of at least one crosslinking agent (XLA) bearing at least two glycidyl functions.

2. The pharmaceutical composition (F) as claimed in claim 1, wherein in the polymer (P), the crosslinking agent (XLA) is chosen from the members of the group consisting of:
ethylene glycol diglycidyl ether of formula (I) 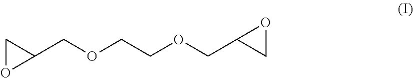 [SMILES: C(COCC1CO1)OCC1CO1]
the compound of formula (II) 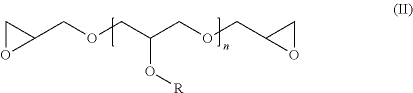
with R representing a hydrogen atom or the glycidyl radical 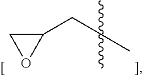
and n representing an integer greater than or equal to one and less than or equal to 10;
1,3-propanediol diglycidyl ether of formula (III) 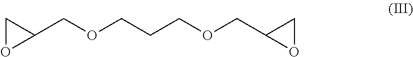 [SMILES: C(COCC1CO1)COCC1CO1]
1,2-propanediol diglycidyl ether of formula (IV) 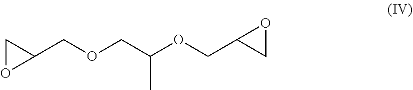
1,4-butanediol diglycidyl ether of formula (V) 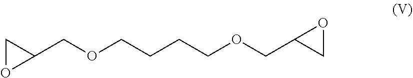 [SMILES: C(CCOCC1CO1)COCC1CO1]
1,2-butanediol diglycidyl ether of formula (VI) 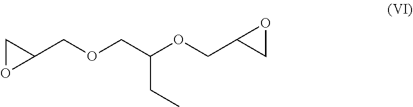
1,3-butanediol diglycidyl ether of formula (VII) 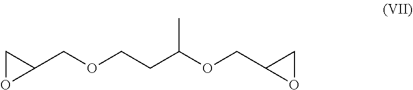
1,6-hexanediol diglycidyl ether of formula (VIII) 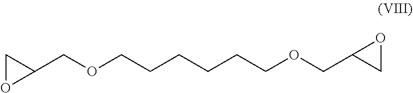 [SMILES: C(CCCOCC1CO1)CCOCC1CO1]
the compound of formula (IX) 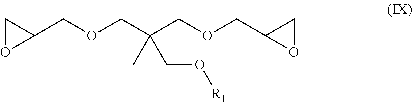
with R1 representing a hydrogen atom or the glycidyl radical 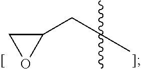 [SMILES: *CC1CO1]
the compound of formula (X) 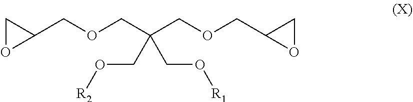
with R1 and R2 independently representing the hydrogen atom or the glycidyl radical 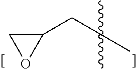 [SMILES: *CC1CO1]
the compound of formula (XI) 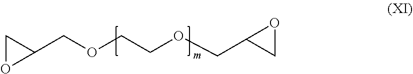
with m representing an integer greater than or equal to 2
the compound of formula (XII) 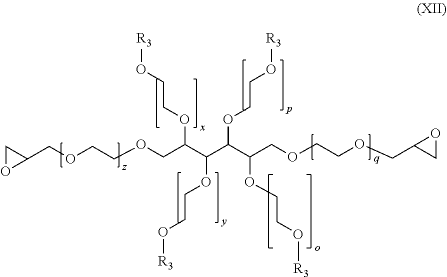
with R3 representing a hydrogen atom or the glycidyl radical 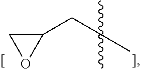 [SMILES: *CC1CO1]
and x, y, z, o, p and q, independently of one another, representing an integer greater than or equal to 2 and less than or equal to 10.

3. The pharmaceutical composition (F) as defined in claim 1, wherein in the polymer (P), per 100 mol % of monomer units derived from partially or totally salified glutamic acid (GA), the crosslinking agent (XLA) represents from 0.5 mol % to 20 mol %, more particularly from 0.5 mol % to 15 mol %, and more particularly still from 0.5 mol % to 12 mol %.

4. The pharmaceutical composition (F) as claimed in claim 1, wherein said polymer (P) additionally comprises a compound of formula (XIV): 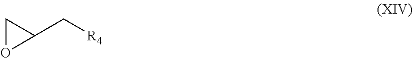
with R4 representing a linear or branched, saturated or unsaturated hydrocarbon-based radical containing from 6 to 22 carbon atoms.

5. The pharmaceutical composition (F) as claimed in claim 4, wherein in formula (XIV), R4 represents a hydrocarbon-based radical chosen from the elements of the group consisting of the octyl, nonyl, decyl, undecyl, dodecyl, tridecyl, tetradecyl, pentadecyl, hexadecyl, heptadecyl, octadecyl, oleyl, linoleyl, linolenyl, eicosyl and dodecosyl radicals.

6. The pharmaceutical composition (F) as claimed in claim 4, wherein in the polymer (P), per 100% of the mass of monomer units derived from partially or totally salified glutamic acid (GA), the compound of formula (XIV) represents from 1% to 50% by mass.

7. The pharmaceutical composition (F) as claimed in claim 1, wherein it comprises, per 100% of its mass, between 0.1% and 10% by mass of said polymer (P).

8. The pharmaceutical composition (F) as claimed in claim 1, wherein the pharmaceutical active principle is chosen from antibacterial agents, antimicrobial agents, antiparasitic agents, antihelminthic agents, anticoccidial agents, anti-Cryptosporidium agents, anti-protozoal agents, antimycotic agents, non-steroidal anti-inflammatory agents,
antiallergic and immunomodulatory agents, analgesic agents, antihistaminic agents, local anesthetic agents, insecticidal agents, antiseptic agents and antifungal agents.

9. A process for preparing a pharmaceutical composition (F) as defined in claim 1, comprising:
a step (A) of preparing a polymer (P), comprising the following substeps:
a) a step of preparing a polar phase (PP) comprising partially or totally salified polyglutamic acid, at least one polar solvent (PS) and at least one crosslinking agent (XLA) comprising at least two glycidyl functions,
b) a step of adjusting the pH of the aqueous solution obtained in step a) to a pH of between 3 and 11,
c) a step of spray-drying the polar phase (PP) resulting from step b) so as to obtain the polymer (P),
a step B) of mixing at least one polymer (P) prepared during step A) with at least one pharmaceutical active principle.

10. The process as claimed in claim 9, wherein wherein in step a), the polyglutamic acid is gamma-polyglutamic acid (PGGA).

11. The process as claimed in claim 10, wherein in step a), all of the monomer units constituting the polyglutamic acid (PGA) are derived from sodium glutamate, potassium glutamate, ammonium glutamate, calcium glutamate, magnesium glutamate or a mixture of these forms.

12. The process as claimed in claim 9, wherein in step a), the polar phase (PP) comprises, per 100% of its mass:
from 5% to 80% by mass of a polymer (P), the polymer (P) is in the form of a pulverulent solid consisting of monomer units derived from partially or totally salified glutamic acid (GA) and of monomer units of at least one crosslinking agent (XLA) bearing at least two glycidyl functions,
from 0.025% to 8% by mass of the least one crosslinking agent (XLA), the crosslinking agent (XLA) is chosen from the members of the group consisting of:
ethylene glycol diglycidyl ether of formula (I) 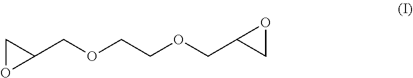 [SMILES: C(COCC1CO1)OCC1CO1]
the compound of formula (II) 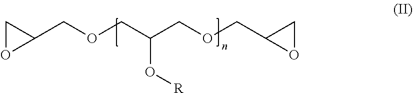
with R representing a hydrogen atom or the glycidyl radical 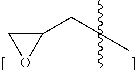 [SMILES: *CC1CO1]
and n representing an integer greater than or equal to one and less than or equal to 10;
1,3-propanediol diglycidyl ether of formula (III) 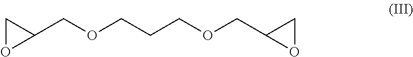 [SMILES: C(COCC1CO1)COCC1CO1]
1,2-propanediol diglycidyl ether of formula (IV) 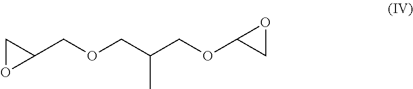
1,4-butanediol diglycidyl ether of formula (V) 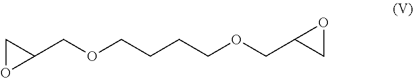 [SMILES: C(CCOCC1CO1)COCC1CO1]
1,2-butanediol diglycidyl ether of formula (VI) 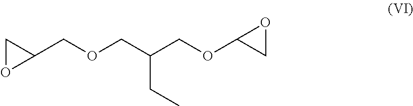
1,3-butanediol diglycidyl ether of formula (VII) 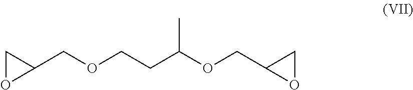
1,6-hexanediol diglycidyl ether of formula (VIII) 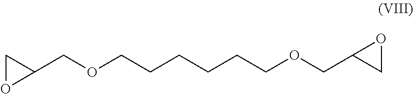 [SMILES: C(CCCOCC1CO1)CCOCC1CO1]
the compound of formula (IX) 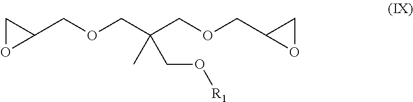
with R1 representing a hydrogen atom or the glycidyl radical 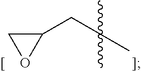 [SMILES: *CC1CO1]
the compound of formula (X) 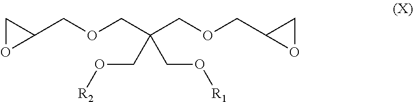
with R1 and R2 independently representing the hydrogen atom or the glycidyl radical 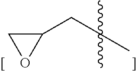 [SMILES: *CC1CO1]
the compound of formula (XI) 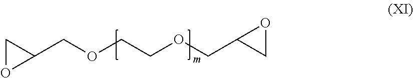
with m representing an integer greater than or equal to 2
the compound of formula (XII) 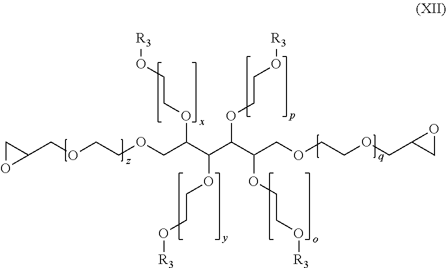
with R3 representing a hydrogen atom or the glycidyl radical 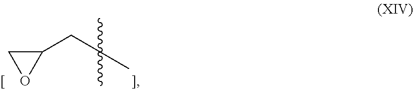 [SMILES: *CC1CO1]
and x, y, z, o, p and q, independently of one another, representing an integer greater than or equal to 2 and less than or equal to 10,
from 12% to 94.975% by mass of at least one polar solvent (PS).

13. The process as claimed in claim 9, wherein wherein in step a), the polar solvent (PS) is chosen from the elements of the group consisting of water, methanol, ethanol, 1-propanol, 2-propanol, isobutanol, tert-butanol, 2-methyl-2-propanol, 1-butanol, 2-butanol, acetone, dimethyl ketone, diethyl ketone, tetrahydropyran, tetrahydrofuran, 2-methyltetrahydrofuran, 1,3-dioxane, 1,4-dioxane.

14. The process as claimed in claim 9, wherein in step a), the polar phase additionally comprises at least one compound of formula (XIV): 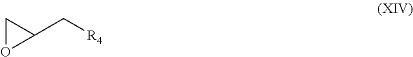
with R4 representing a linear or branched, saturated or unsaturated hydrocarbon-based radical, optionally containing at least one hydroxyl function and containing from six to 22 carbon atoms.

15. The process as claimed in claim 14, wherein R4 represents a hydrocarbon-based radical chosen from the elements of the group consisting of the octyl, nonyl, decyl, undecyl, dodecyl, tridecyl, tetradecyl, pentadecyl, hexadecyl, heptadecyl, octadecyl, oleyl, linoleyl, linolenyl, eicosyl and dodecosyl radicals.

16. The process as claimed in claim 14, wherein the content of compound of formula (XIV) in the polar phase (PP) is, per 100% by mass of said polar phase (PP), between 0.05% and 35% by mass, it being understood that the sum of the proportions by mass of the polyglutamic acid, of the crosslinking agent (XLA), of the polar solvent (PS) and of the compound of formula (XIV) is equal to 100%.

17. The process as claimed in claim 9, wherein step c) is carried out using an atomizer employing an air flow and a nozzle.

18. The process as claimed in claim 17, wherein the inlet temperature of the air flow is between 80° C. and 180° C., preferably between 100° C. and 170° C.

19. The use of said polymer (P) as defined in claim 1 as thickening and/or emulsifying and/or stabilizing agent for a pharmaceutical composition for topical use.
